(19) United States
(12) Patent Application Publication
(10) Pub. No.: US 2008/0003140 A1
(43) Pub. Date: Jan. 3, 2008

(54) TITRATION SYSTEM

(75) Inventors: Jiandong Di, Zurich (CH); Rene Rissi, Bertschikon (CH); Jurgen Zarske, Uerikon (CH); Christof Bircher, Hinwil (CH); Antonio De Agostini, Bern (CH)

Correspondence Address: STANDLEY LAW GROUP LLP, 495 METRO PLACE SOUTH, SUITE 210, DUBLIN, OH, 43017, US

(73) Assignee: Mettler-Toledo AG, Greifensee (CH)

(21) Appl. No.: 11/843,244

(22) Filed: Aug. 22, 2007

Related U.S. Application Data
(63) Continuation of application No. PCT/EP2006/062736, filed on May 30, 2006.

(30) Foreign Application Priority Data
Jun. 29, 2005 (EP) 05107037.3

(51) Int. Cl. G01N 33/48

(52) U.S. Cl. 422/68.1; 436/163

(57) ABSTRACT

A titration system has a base module, a controller unit containing a processor with an operating program, and at least two components that are in releasable electronic connection to the controller unit. The first component is an interchangeable dosage-metering unit, and the further components could include: a further dosage-metering unit, a sensor, a plug-in card, and a titration module. Each of the components has a means for identifying it, using a unique identifier that is transmitted to the controller unit. The titration system is designed to automatically register the adding and/or removing of components, and to automatically update a virtual configuration of the titration system that is stored in the operating program while the system is in an operation-ready state, especially during an analysis.

### CROSS-REFERENCE TO RELATED APPLICATIONS

This application is a continuation under 35 USC 365 of PCT/EP06/062736, filed 30 May 2006, which claims foreign priority under 35 USC 119 from European patent application 05107037, filed 29 Jun. 2005, the content of which is incorporated by reference as if fully recited herein.

### TECHNICAL FIELD

The invention relates to a modular titration system.

### BACKGROUND OF THE ART

Automated titrators or titration systems are used primarily in chemical laboratories to perform titrimetric analyses of mostly liquid samples in a simple and efficient manner.

The titration systems of the known state of the art generally have one or more dosage-metering units that are connected to a controller unit. Each dosage-metering unit generally includes a burette which is arranged in the dosage-metering unit, and the dosage-metering unit is connected to a supply bottle which contains a titration agent such as for example a base or an acid of a known concentration. A sample to be analyzed is held in a suitable sample container, and prescribed quantities of a titration agent are added to it in steps. For the monitoring of changes which occur as a result of adding titration agent, one or in some cases several sensors are in contact with the sample. These sensors may include for example temperature sensors, pH sensors, conductivity sensors, sensors for a Karl Fischer titration, or other known sensors.

The titration systems of the known state of the art are generally designed to allow the installation of a set number of dosage-metering units into a base instrument, wherein the latter has only limited expansion capabilities. For example, commonly-owned German application DE 39 13 632 A1 discloses a titration system in which several dosage-metering units can be arranged on one base instrument.

A dosage-metering device is disclosed in published European application EP 1 103 793 A1, with a dosage-metering unit that is connected to a supply container. The supply container has an electronic tag in the form of a read/write memory device, which contains data regarding the contents and the supply volume remaining in the container, wherein these data have to be entered or reset again with each change of the supply container.

A measuring- or dosage-metering device is disclosed in published European application EP 0 994 350 A1 which includes a sensor and a supply container with a calibration reference solution, wherein the sensor and the container can exchange calibration data with a base instrument.

### SUMMARY OF THE INVENTION

The primary characteristics desired in a titration system are that it should be particularly easy, simple and nevertheless safe to operate and should at the same time facilitate the compliance with the regulatory standards as mandated by law. It is therefore particularly advantageous for a user, if a titration system can be changed at any time in a quick and simple manner and adapted to the individual needs of the user. The titration system, according to the appended device claims, represents a particularly user-friendly system, and a method of operating this titration system is set forth in the appended method claims.

A titration system includes a base module, a controller unit containing a processor with an operating program, an interchangeable first dosage-metering unit, and at least one component with a releasable electronic connection to the controller unit. Such a component can be in particular a further dosage-metering unit, a sensor, a plug-in card, and/or a titration module. The first dosage-metering unit and the component each have a means of recognition (or identification) with a unique identification that can be transmitted to the controller unit. The titration system is designed in such a way that in an operation-ready state, and in particular during an analysis, the system has the capability to automatically register the adding and/or removing of the first dosage-metering unit and/or the component, and to automatically update a configuration of the titration system which is stored in the operating program.

A titration system of this kind is very user-friendly, because it allows the user to perform a changeover of the system, for example in preparation for a subsequent analysis, by adding and/or removing a dosage-metering unit and/or a component not only in the switched-off condition, but also in the operative condition and even while an analysis is in process. The controller unit is equipped with the capability to register the addition and/or the removal of a dosage-metering unit and/or a component and to initiate an update of the currently stored system configuration in the operating program without affecting or making it necessary to stop a currently running analysis. Based on the information transmitted from the means of recognition, it is not only possible to register the removal and/or the addition of a component and/or a dosage-metering unit, but also the arrangement of the different parts of the titration system in relation to each other. Thus, the means of recognition serves the purposes of uniquely identifying the component and/or dosage-metering unit to which it is attached and also to locate it, i.e. to determine its position in the titration system.

Each of the means of recognition includes a unique, unchangeable identification, for example in the form of a serial number through which the dosage-metering unit and/or the component can be unequivocally identified.

The titration system preferably has a modular design concept, which makes it possible for the user to assemble a titration system that is adapted to the user's individual needs, and to expand the system as needed by supplementing the titration system with an individually determined number of components and/or dosage-metering units. The components as well as the dosage-metering units are designed so that they can be connected to and/or removed from the controller unit during operation. Subsequent to being connected and/or removed, the components and/or dosage-metering units can be recognized and initialized automatically by the controller unit.

The controller unit is equipped with at least one processor, a main board, a data interface, a storage memory and a bus system, preferably a CAN (Controller Area Network) bus system.

A component can for example be a stirrer, a sample changer, a supply container, an external processing unit and/or an analyzer- or peripheral instrument such as a balance or a printer, wherein the titration system is designed so that it can include as well as control one or more components of the same kind and/or of different kinds, and/or dosage-metering units. The term “peripheral instrument” in this context means any instrument that is capable of being connected to the titration system.

The means of recognition of the dosage-metering unit and/or of the component include a non-volatile read/write memory into which system-specific and/or user-specific data can be written. The writing of the data can be done either at the factory and/or by the user. Preferably, the storage memory includes at least one secure, for example password-protected and/or write-protected portion which cannot be changed by the user. In addition to a unique serial number, the system-specific data can also include information regarding the parameters of the dosage-metering unit and/or the component, such as the volume, an expiration date, the measurement tolerance of a dosage-metering unit or of the burette contained in the dosage-metering unit, and/or information regarding the certification of the dosage-metering unit and/or the component. The user-specific data can include, among other things, information about the titration agent being used, such as its chemical composition, lot number, titer concentration and/or the time of the last verification test of the titer, as well as other information and data that are relevant for the user. The system-specific as well as the user-specific data can be exchanged between the means of recognition and the controller unit and can be adapted if needed.

With preference, the first dosage-metering unit and/or the component are connected electronically as well as mechanically to the titration system, wherein the electronic connection is designed so that the dosage-metering unit and/or the component can communicate with the regulating- and/or control unit. The communication can be unidirectional or bidirectional, with the possibility for bidirectional communication normally being provided.

In a preferred embodiment, the means of recognition includes a first identifier element which is connected to the first dosage-metering unit or to the component and is equipped with the capability for wireless communication of the unique identification to a second identifier element which is connected to the titration system. A wireless communication between the titration system and the dosage-metering unit and/or the component can be realized for example with RFID (Radio Frequency Identification) technology, where one of the identifier elements is designed as an RFID transponder and the other as RFID receiver. RFID transponders are also called RFID tags and are often distinguished already at the factory by a unique identification in the form of a serial number as well as by a non-volatile read/write memory. The known state of the art includes active and passive RFID transponders, with passive transponders that need no electric power supply of their own being used preferably in combination with an active sender- and/or receiver unit.

In a preferred embodiment, the means of recognition is an active means of recognition which in addition to a read-/write memory and a unique identification includes a micro-controller and has the capability for active communication with the controller unit.

The configuration of a modular titration system as taught herein is extremely flexible, which makes it possible to electronically connect the first dosage-metering unit and/or the component directly and/or by way of at least one further component to the controller unit. At least one dosage-metering unit and/or at least one component can be connected in series or parallel—so to speak—to the controller unit.

The controller unit has at least one interface for the transmission of data and/or power. The dosage-metering unit and/or the components can be electronically connected to the controller unit by way of a suitable interface. Depending on the dosage-metering unit and/or component to be controlled, the at least one interface can have the form of an RS232 interface, a USB interface, an Ethernet interface, a TTL interface, a CAN bus connection, a current- and/or voltage connection, a parallel port, a serial port, and/or a specific connection for a sensor, a measuring probe and/or a peripheral instrument. This enumeration represents only a small part of the possible interfaces and/or connections. It is possible in principle to realize all state-of-the-art interfaces and connections. Normally, a titration system as taught herein is equipped with several different kinds of interfaces.

The base module of the titrations system is connected to the controller unit, which is represented either by a part of the base module or can also be realized as a separate instrument. The controller unit, the base module, as well as the at least one component, in particular a titration module, are designed in such a way that data and/or power can be transmitted between them in either direction, with the communication taking place preferably by means of a bus system. Particularly advantageous are CAN bus systems. A CAN bus system is a state-of-the-art bus system which allows a transfer of data as well as power and in addition provides automatic recognition of the components connected by this bus system. Consequently, it is possible with a bus system that for example a titration module is connected to the titration system, recognized and initialized while the system is in operation without the need for restarting and/or re-initializing the titrations system, the controller unit and/or an application software or operating program. The titration program can be expanded during operation by further components such as titration modules and/or other peripheral instruments which, dependent on the connection being used, can be recognized automatically. The titration modules are preferably not equipped with their own separate power supplies, but are supplied with power as well as controlled by the controller unit through a suitable bus system designed for the transfer of data and power.

The titration module and/or the base module in a preferred embodiment includes a docking device and a drive source for a dosage-metering unit. In particular the base module can have one or more docking devices as well as one or more drive couplings for one or more dosage-metering units. This is an advantageous arrangement in that the base module alone already represents a functional titration system which can be combined and/or expanded with one or more titration modules or other components.

The dosage-metering unit is preferably equipped with a state-of-the-art piston burette which has a glass cylinder of a defined volume. In this kind of burette, the piston is moved in the glass cylinder in a controlled manner by a drive mechanism, so that the burette or more specifically the glass cylinder can take in or dispense a specified volumetric quantity of the titration agent.

The titration module in a preferred embodiment has at least two titration modules arranged side by side, which are electronically connected to the controller unit. The titration modules are mechanically connected to each other in such a way that their docking devices form a common channel in which at least one dosage-metering unit can be seated and/or slid along. The common channel makes it easy to insert and/or remove a dosage-metering unit. With preference, a titration system designed to operate with a plurality of dosage-metering unit is in addition equipped with at least one docking device that is designed as a parking and/or exchange position.

An embodiment of the titration system includes a display which is electronically connected to the controller unit and which can also serve as an entry unit, depending on the design. The display allows for example to present a view of the configuration of the titration system as well as analytical procedures and/or the results of analyses. With a combined display- and entry unit, one can enter, e.g. system- and/or user-specific data, analytical procedures and/or control commands.

In a preferred embodiment, the controller unit is arranged in the base module, although a titration system can also work together with an external controller unit, which may in some cases provide the display- and/or entry unit.

To allow user-specific data and/or control commands and/or control programs to be entered, the titration system is equipped with an input- and/or output unit. The input- and/or output unit can include for example a terminal that is connected to the controller unit, a keyboard and/or an external computer. The input- and/or output unit can be designed as a touch screen, or as a monitor with an integral keyboard, or as a part of an external computer. In addition, the titration system can include, e.g. a printer, an internal memory or an external memory for the output or for the storage of the data. The controller unit can be arranged in an external input- and/or output unit, for example in an external computer, which can be connected to the titration system directly as well as through any kind of data interface, for example USB, CAN bus, or Ethernet. In the latter case it is advantageous if the base module includes at least a part of the controller unit, which can in this case be remote-controlled from the external computer.

A method for the operation of a titration system with a base module, a controller unit including a processor with an operating program, an interchangeable first dosage-metering unit, and at least one component with a releasable electronic connection to the controller unit—the latter being in particular a further dosage-metering unit, a sensor, a plug-in card, a titration module, a stirrer, a sample changer, a supply container, an external processing unit and/or an analyzer- or peripheral instrument, with the first dosage-metering unit and the component each carrying a means of recognition with a unique identification—has the distinguishing feature that after a change has been made in an existing configuration of the titration system, in the operation-ready condition and/or while an analysis is in process, the controller unit initiates an update of the configuration data of the titration system that are stored in the operating program. The titration system can be changed by adding and/or removing the first dosage-metering unit and/or by adding and/or removing the component. The first dosage-metering unit and the component each have a means of recognition, whose unique identification can be transmitted to the controller unit. Furthermore, the controller unit can through interrogation detect the presence and/or absence of the first dosage-metering unit and/or the component. Based on the identification that has been transmitted or acquired by interrogation, as well as based on a positive and/or a negative response to the interrogation, in particular based on a repeated positive and/or negative response, the controller unit can register the added and/or removed first dosage-metering unit and/or the component and can trigger an update of a configuration of the titration system that is stored in the operating program.

The controller unit is alerted regarding the absence as well as regarding the addition of a dosage-metering unit and/or a component and based on this information triggers an update of the configuration data of the titration system that are stored in the operating program. The required identification as well as the required information can be transmitted actively from a means of recognition to the controller unit, or they can also be received and registered as positive or negative responses to an interrogation by the controller unit.

After the controller unit has registered an absence and/or an addition of a dosage-metering unit and/or a component, but prior to the actual updating of the configuration data of the titration system that are stored in the operating program, a notice with a decision request is initiated and presented to the user by means of a display. The user can accept the configuration or reject it for example if the component and/or the dosage-metering unit were added in a wrong position. Following a positive response by the user, the updating of the set of configuration data of the titration system stored in the operating program is executed. With the inventive system and method of operation, the addition and/or removal of a dosage-metering unit and/or a component as well as the updating can be performed in the operation-ready condition of the titration module as well as during an analysis process without the need to restart the titration system and/or the controller unit. An attempt to remove the dosage-metering unit and/or the component required in a currently running analysis triggers an acoustic and/or optical error message, in which case the removal of a required dosage-metering unit and/or component is made difficult and/or prevented by suitable mechanical locking devices. The unique identification can be transmitted in a wire-bound and/or a wireless mode between the controller unit and the first dosage-metering unit and/or the component.

As a check, the controller unit can repeat the interrogation about the configuration of the titration system at given time intervals. As soon as a change of the configuration is detected, an accept/reject decision request is presented to the user. It is advantageous if this decision request is generated only after the same interrogation response has been received a certain number of times by the controller unit.

The operating program is designed with the capability to control and/or regulate the operating procedures of the titration system. The term “operating procedures” in this context encompasses for example different actuating commands, operating programs, as well as the acquiring and processing of measurement data.

### BRIEF DESCRIPTION OF THE DRAWINGS

Different embodiments of the titration system are described hereinafter as examples with references to the drawings, wherein:

FIG. 1 is a schematic three-dimensional view of a titration system with a base module, two titration modules, three dosage-metering units with attached supply containers, an entry- and/or output unit and a titration arm with sample container, stirrer, sensors and supply conduits;

FIG. 2 is a schematic three-dimensional view of a titration system with a base module, two titration modules, and two dosage-metering units;

FIG. 3 is a schematic view directed at the back side of another titration system, this system having a base module, four titration modules, a total of four dosage-metering units, and two titration arms;

FIG. 4 is a top view of an enlarged detail of a docking device with a dosage-metering unit slid partway into place, with the docking device and the dosage-metering unit partially cut away for a clearer view, as indicated by the hand-drawn lines; and

FIGS. 5a to 5e are greatly simplified schematic top views of a titration module and/or a base module with differently configured docking devices.

### DETAILED DESCRIPTION OF A PREFERRED EMBODIMENT

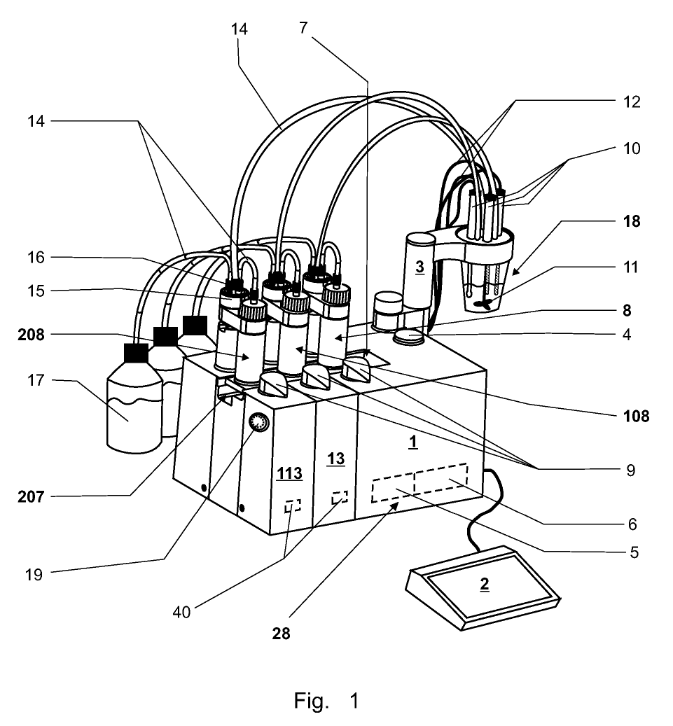

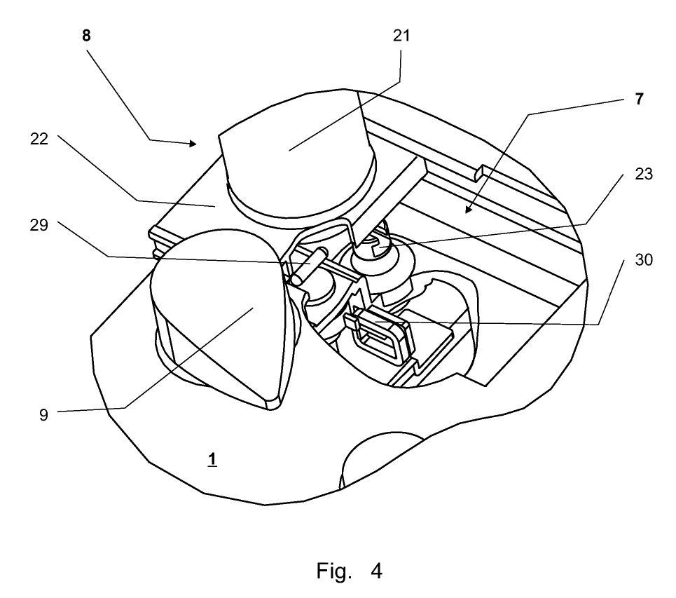

A preferred embodiment of a modular titration system is shown in FIG. 1. This titration system includes a base module 1 and a controller unit 28 which is arranged inside the base module 1 and whose presence is only indicated by a broken outline. The controller unit 28 includes at least one processor 5, a read/write memory 6, as well as an operating program. Connected to the controller unit 28 is an entry- and/or output unit 2 for data and information, which is preferably equipped with a touch screen. The base module 1 further includes a titration arm 3 and a connector socket 4 for a further titration arm, a docking device 7 (see FIG. 4 for details) for a dosage-metering unit 8, as well as a turn handle 9 for locking the dosage-metering unit 8 into place. Preferably, the mechanical locking device 9 is designed for manual operation and immobilizes the sliding base of the dosage-metering unit 8 in the docking device 7, so that the dosage-metering unit cannot be inadvertently removed for example while an analysis is in process. The term “analysis” is used here for example in the sense of a titration.

The titration arm 3 in this embodiment holds an interchangeable sample container 18, with several sensors 10 (three are shown) and a stirrer 11 descending into the sample container from above. The sensors are held by the titration arm 3, and each of the sensors is connected for example by a suitable cable 12 and an interface to the controller unit 28 which is arranged in the base module 1. Each of the sensors 10 as well as the stirrer 11 has a means of recognition (not visible in the drawing), which contains a unique identification as well as further information regarding the stirrer 11 or the individual sensor 10.

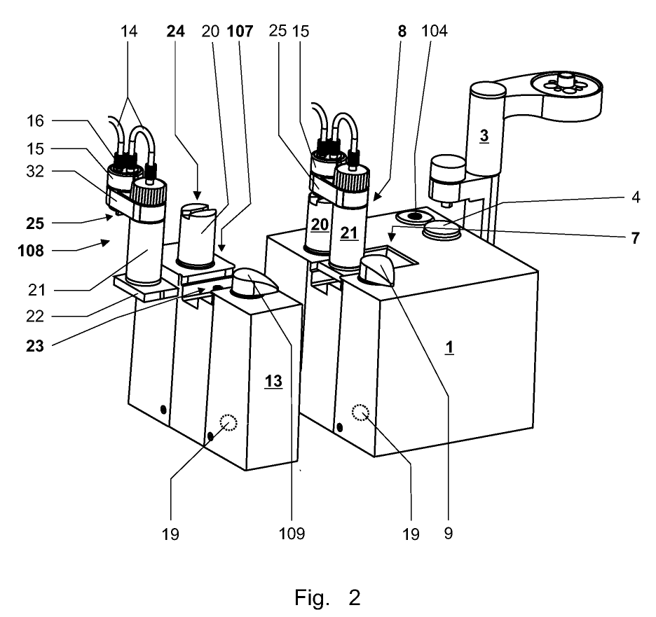

The dosage-metering unit 8 is releasably connected to the docking device 7 and can be locked in place in an operating position by means of the locking device 9. The locking device 9 is preferably configured for manual operation, by turning the turn handle from an open into a closed position (also see FIG. 2). The dosage-metering unit 8 includes an interchangeable burette (not explicitly shown here) which is driven by a drive mechanism (also see FIG. 4) that is arranged in the base module 1 and controlled by the controller unit 28. The burette which is part of the dosage-metering unit 8 is preferably a piston burette. It is possible to use dosage-metering units with burettes of different volume capacities. The dosage-metering unit as well as the drive mechanism belong to the known state of the art and will therefore not be described in further detail.

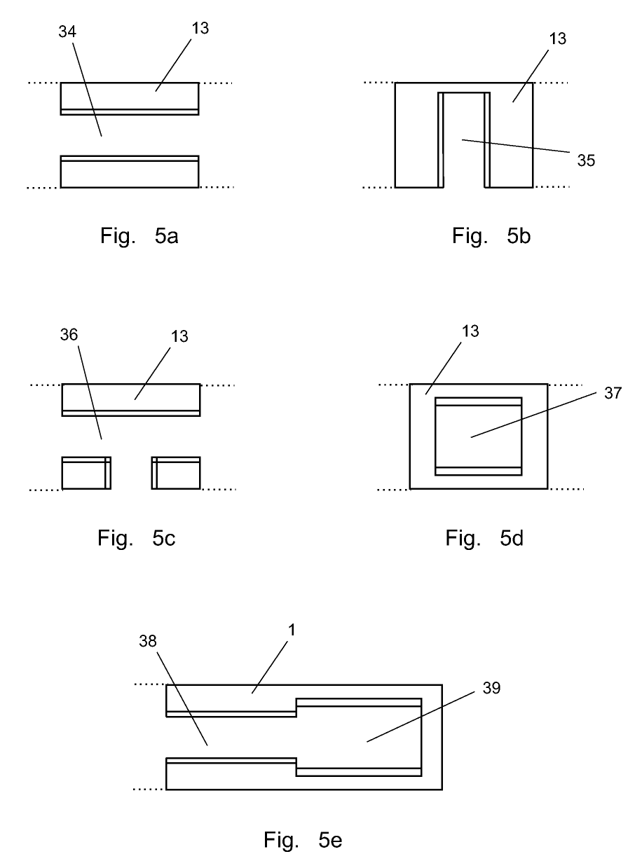

The docking device 7 is formed in the base module 1 as a recessed channel designed to receive a dosage-metering unit 8 and to couple it to the drive mechanism. The docking device 7 is configured in such a way that it can in addition serve as a parking position and/or exchange position for one dosage-metering unit (see also FIG. 5e). This is the reason why the docking device 7 is more than twice as long as the sliding base of the dosage-metering unit 8.

The base module 1 is connected on one side to a first titration module 13. This connection can be either a permanent connection or a releasable connection. The connection is considered as permanent if the base module 1 and the first titration module 13 share for example a common housing, or if their housings are solidly connected to each other for example by suitable connecting means such as screws, rivets, and/or by adhesive bonding.

A releasable connection between the base module 1 and the titration module 13 can be achieved for example with any kind of plug-in connection. Preferred is a releasable magnetic connection between the base module 1 and the titration module 13, particularly in combination with meshing projections and indentations in the housings of the base module 1 and the titration module 13. To provide a releasable magnetic connection, the base module 1 and the titration module 13 are each equipped with at least one permanent magnet oriented so that the magnets attract each other if the base module 1 and the titration module 13 are brought closer than a certain distance to each other. The strength of the magnets is designed so that the titration module 13 will safely adhere to the base module 1 but can also be removed again without requiring an auxiliary device.

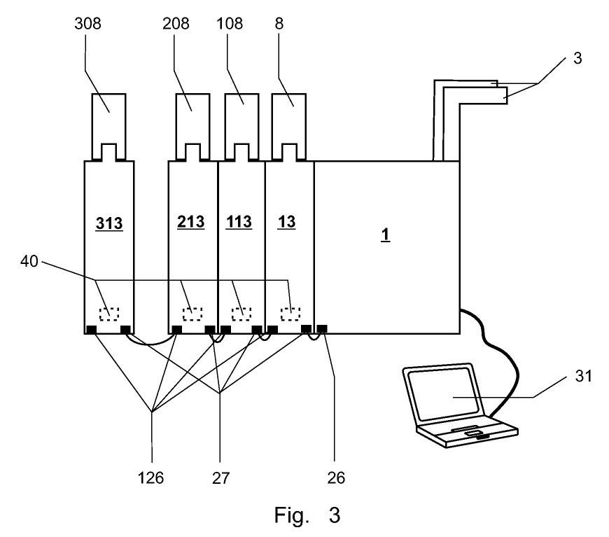

However, the magnetic connection only serves in a mechanical sense to hold the titration system together and to give it stability. In addition, the titration module 13 is connected through suitable cables by way of a bus system to the base module 1 to transmit data and/or power between the controller unit 28 in the base module 1 and the titration unit 13. The preferred means for transmitting data and/or power is a CAN bus system (see also FIG. 3).

The titration module 13 carries a means of recognition 40 which cooperates with the bus system and has the capability to transmit through the bus system a unique identification and further information about the titration module 13 as well as the position of the titration module to the controller unit 28.

Like the base module 1, the titration module 13 includes a dosage-metering unit 108 arranged in a docking device, a locking device 9, and a drive mechanism for the burette that is arranged in the dosage-metering device 108. The docking device of the titration module 13, which is hidden from view by the dosage-metering device 108, takes up the entire horizontal length (in reference to the view shown in FIG. 1) of the titration module 13.

The titration module 13 is oriented relative to the base module 1 in such a way that the docking device of the titration module 13 meets up and forms a common channel with the docking device 7 of the base module 1, so that a dosage-metering unit 8, 108 can be either slid from the left side (in reference to the drawing) into the docking device of the titration module 13 or pushed through the docking device of the titration module 13 into the docking device of the base module 1, and that it can also be taken out of the titration system again in the opposite direction.

Every dosage-metering unit 8, 108 carries a first identifier element, in this case an RFID tag, which interacts with a second identifier element, in this case an RFID receiver, wherein one of each is connected to each docking device. The design, arrangement and function of the identifier elements will be explained hereinafter in more detail in the context of FIG. 4. The identifier elements work in such a way that when a dosage-metering unit 8, 108 is in contact with a docking device, it will be automatically recognized and initialized by a software program that is implemented in the controller unit 28, without the need to restart and/or re-initialize the software program, the controller unit 28 and/or the entire titration system. The dosage-metering units can be joined to the titration system and/or removed from it while the latter is operating and running.

A further titration module 113 is arranged adjoining the titration module 13 on the opposite side from the base module 1. The titration module 113 is connected to the titration module 13 preferably likewise in a releasable manner. Each titration module 13, 113 is equipped for this purpose with at least one permanent magnet 19 on each of two opposite sides, to form a releasable magnetic connection.

The titration module 113 is identical to the titration module 13, with the docking device 207 being visible here with the dosage-metering device 208 seated in it. The titration module 113 in this embodiment is connected to the titration module 113 in such a way that the docking device 7 of the base module 1, the docking device of the titration module 13 and the docking device 207 of the titration module 113 form a continuous channel. Thus, at least one dosage-metering device can be slid from the left (in reference to the drawing) into the docking device 207 and pushed along and locked into any of the docking devices. The dosage-metering device can be separated from the titration system either in the reverse way and/or by way of the base module 1 which has a docking device 7 configured to serve also as a parking- and/or exchange position.

The dosage-metering unit 208 is likewise equipped with an interchangeable burette and is connected by way of suitable hoses 14 and or conduits of a chemical-resistant material to a distributor 15 which belongs to the dosage-metering unit 208. This distributor 15 has several hose connectors 16, three of which are shown here, and is connected through separate hoses 14, respectively, to a supply container 17 containing a titrating agent, to the burette arranged in the dosage-metering unit 208, and to the sample container 18. The distributor 15 with the three hoses 14 shown here performs the function of a switching valve which can be switched to connect the burette either to the supply container 17 or to the sample container 18, so that the burette can aspirate or dispense titration agent. The preferred types of burettes to be used here are piston burettes with a glass cylinder of defined volume which interact with a drive mechanism that is arranged in the titration module 113. In the interest of clarity, these features are identified in FIG. 1 with reference symbols only for the dosage-metering unit 208, with the further dosage-metering units 8,108 being of analogous configuration.

The modularity of the titration system is explained in more detail through the example of the embodiment shown in FIG. 2. In this representation, several elements of the titration system are not yet connected to the latter but are shown in an exploded view for better clarity.

The titration system consists of the base module 1 and a titration module 13, each of which has a docking device 7, 107 and a dosage-metering unit 8, 108. The base module 1 has a titration arm 3 which is shown here without sensors, sample container or stirrer and is not yet connected to the titration system, so that a covered connector socket 4 as well as an open connector socket 104 for a titration arm can be seen. Instead of a titration arm 3, one could also connect a state-of-the-art stationary titration stand with a sample container and/or a sample changer to the base module 1. For a description of the base module 1 and the titration arm 3, the reader is referred to the description of FIG. 1.

FIG. 2 shows the base module 1 separated from the titration module 13, so that some of the places for the magnets 19 can be seen. Magnets 19 are arranged inside one side wall of the housing of the base module 1 and inside two opposite sidewalls of the housing of the titration module 13, drawn in dotted lines, with only one possible arrangement for placing the magnets 19 being shown here. There are other possible places where magnets 19 or other mechanical connecting means can be arranged on or inside the housing.

The dosage-metering unit 108 is not yet connected to the titration module 13, so that the channel-like docking device 107 can be seen as well as a securing column 20 in which a guide groove 24 is formed.

The dosage-metering unit 108 consists of a sliding base 22 designed to cooperate with the docking device 107 so that it can be slid into or through the latter, with a substantially cylindrical housing 21 holding an interchangeable burette and with a holder 32 that connects the burette housing 21 to the distributor 15. Arranged inside each of the dosage-metering units 8, 108 is a first identifier element which interacts in such a way with a second identifier element that is included in each of the docking devices 7, 107 that a dosage-metering unit 8, 108 is automatically recognized when it is slid into a docking device 7, 107 (also see FIG. 4).

The docking device 107 is designed so that a dosage-metering unit 8, 108 can be slid into it from either side. Thus, the docking device 107 takes up the entire length (in reference to the drawing) of the titration module 13. About midway, the docking device 107 has a drive shaft which cooperates with the drive mechanism that is arranged in the titration module 13. An identical drive mechanism with a drive shaft is also arranged in the base module 1, but is covered in this view by the dosage-metering unit 8. The drive shaft 23 cooperates with the burette that is arranged in the dosage-metering unit 108, if the dosage-metering unit 108 is seated in the docking device 107 and is secured by means of the locking device 109 in the docking device 107. The locking device 9 arranged on the base module 1 is shown in the locked position, and the locking device 109 on the titration module 13 is shown in the open position.

Besides the docking device 107, the dosage-metering unit 108 is also constrained in its movement and locked in the operating position by a rotatable securing column 20. The securing column 20 has a groove 24 which can work together with a projection 25 that is arranged on the holder 32 of the dosage-metering unit 108.

Each of the dosage-metering units 8, 108 is secured by the manually releasable locking device 9 as well as by the rotatable securing column 20. The dosage-metering device 8, 108 can be used only if it is in operative contact with the locking device 9, 109 and with the securing column 20. In the operating state, the securing column 20 is turned by 90° relative to its orientation as shown in FIG. 2, so that the groove 24 is aligned perpendicular to the docking device 107. The turning of the securing column 20 causes a turn of the projection 25, whereby the distributor is caused to unlock the conduits 14, so that the titration agent can be transferred from the supply container to the dosage-metering unit and from there to a sample.

FIG. 3 shows a schematically simplified rear view of a further embodiment of a titration system which includes a base module 1 with 2 titration arms 3, an external entry and/or output unit 31 in the form of a computer, as well as four titration modules 13, 113, 213, 313, each with a dosage-metering unit 8, 108, 208, 308. The base module 1 is connected through suitable means such as magnets to the first titration module 13 which, in turn, is connected by way of the titration module 113 to the titration module 213. The titration module 313 is spatially separated from the other titration modules and has only an electrical connection by way of the titration modules 13, 113, 213 to the base module 1.

In this embodiment, only the titration modules 13, 113, 213, 313, but not the base module 1, have docking devices for dosage-metering units 8, 108, 208, 308. The titration system is connected to an external computer which serves as entry- and/or output unit 31. The external entry- and/or output unit 31 is connected through a cable directly to the titration system, wherein the controller unit in this example is integrated in the external entry- and/or output unit 31.

The dosage-metering units 8, 108, 208, 308, the titration modules 13, 113, 213, 313, and the base module 1 are substantially identical to those already shown in FIGS. 1 and 2, and are therefore not explained again here.

The base module 1 has at least one connector plug 26, and each titration module 13, 113, 213 has a connector socket 27 as well as a connector plug 126. The connectors 26, 126, 27 are preferably configured as a CAN bus system. CAN-bus systems allow the transfer of data and power as well as the automatic recognition and initialization of a titration module 13, 113, 213, 313 which has been added to or removed from the titration system during operation. To allow automatic recognition, each titration module 13, 113, 213, 313 has a means of recognition 40 with a unique identification. Besides a unique identification for example in the form of a serial number or an identification chip, the means of recognition 40 includes a non-volatile read/write memory as well as a microcontroller.

To transfer data and/or power from the controller unit in the base module 1 to the four titration modules 13, 113, 213, 313 and in particular to the drive mechanism built into each of the titration modules 13, 113, 213, 313, a connector plug 26, 126 is in each case connected to a connector socket 27 in such a way that the base module 1 and the titration modules 13, 113, 213, 313 are connected in series. The circuit is laid out in such a way that the transfer of data and/or power is directed in a loop through the titration modules 13, 113, 213. The CAN bus system serves not only for the transfer of data and/or power from the controller unit to the titration modules 13, 113, 213, 313, but also for the data return from the titration modules 13, 113, 213, 313 to the controller unit.

The controller unit has several PCB (Printed Circuit Board) sockets for different PCBs to be plugged in. One of the sockets is provided for a main board which contains the processor, while the further sockets can be used to install different plug-in boards serving for the control of, e.g., sensors, sample changers, stirrers, pumps, balances, the entry- and/or output unit, a printer, or other peripheral instruments. The plug-in cards can have for example an RS232 interface, USB connections, CAN bus system connections, parallel ports, serial ports, as well as special process connections and other state-of-the-art connections.

The concept of using a CAN bus system for connecting the titration modules 13, 113, 213, 313 to the base module 1 and to each other in combination with an identifier element associated with each component ensures a particularly user-friendly way of assembling individual titration systems.

Based on the means of recognition associated with each component and/or each dosage-metering unit, it is possible for example for a dosage-metering unit to be connected to any docking device during operation of the titration system and even while an analysis is in process, and to be automatically recognized in a way that allows the type of dosage-metering unit as well as its position in the titration system to be registered.

FIG. 4 represents a detail view directed from above at a docking device and a dosage-metering unit of a base module or of a titration module. In FIG. 4, the dosage-metering unit 8 is inserted only partway into the docking device 7, and the locking device 9 is in the open position. The elements are shown partially in a cut-away view to allow a look into the interior.

The docking device 7 shown in the drawing represents a part of a base module 1 of which only a part of the housing can be seen here. It should be noted that the docking device 7 of the base module 1 is of a substantially identical design as the docking device of any of the titration modules.

Among the components of the dosage-metering device 8, one recognizes in this representation primarily the burette housing 21 and the sliding base 22 which works together with the docking device 7. Arranged inside the sliding base 22 is a first identifier element 29, while a second identifier element 30 is arranged below the docking device 7 in the interior of the housing of the base module 1, wherein the positions of the first and second identifier elements 29, 30 can also be exchanged. With preference, the first identifier element 29 is configured as a passive identifier element, and the second identifier element 30 is configured as an active element, so that at least the first identifier element 29 does not need a power supply of its own. This is particularly advantageous, since a dosage-metering unit equipped with a piston burette can be powered by means of a drive shaft 23 that is arranged in the docking device 7, so that the dosage-metering unit does not need an electric power supply of its own.

An identifier element, preferably the first identifier element 29 which is connected to the dosage unit 8, includes a non-volatile read/write memory in which a unique identification number is stored among other things. In addition, data specific to the dosage-metering unit 8 as well as user-specific data can be written into the read/write memory. The data of the dosage-metering unit 8 are stored in a specially secured area of the memory with read-only access for the user. These data, which can include data such as the burette volume, the tolerance of the burette volume, the serial number of the burette, the time of the last and/or the next certification, can be changed only by individuals who have been given read/write access, for example in connection with a maintenance service or a recertification. The user is given read- and write-access to another memory area where the user can store information specific to his application. This user-specific information includes for example the name, the concentration, the lot number, the expiration date, and the titer of the titration agent being used, the date of the last and/or the next verification check of the titer, as well as the preferred filling speed, and in some cases also rules and deadlines which for example preclude the use of the titration agent beyond its expiration date.

The entry of this information occurs for example through an entry- and/or output unit that is connected to the titration system directly or through an Ethernet connection, when the dosage-metering unit 8 is connected to the docking device 7.

In a preferred embodiment, the first identifier element 29 is a passive RFID transponder and the second identifier element 30 is a corresponding active sender- and/or receiver unit which is connected to the controller unit in the base module 1 either directly or by way of the CAN bus system.

Passive RFID transponders and active sender- and/or receiver units corresponding to them are available with different distance ranges from a variety of suppliers such as Infineon, among others. In this example of an embodiment, it is particularly advantageous to use identifier elements that operate within near-distance range, i.e. within 10 to 30 cm, in a frequency range of about 100 to 150 kHz and preferably between 120 and 130 kHz. The second identifier element 30 which is configured as a sender- and/or receiver unit is connected either directly to the controller unit that is arranged in the base module 1 or by means of a connection serving for the transfer of data and/or power between at least one titration module and the base module 1. Among other things, the second identifier element 30 is supplied with power and is also controlled through this connection. The second identifier element 30 emits a signal in the form of electromagnetic waves. If a passive first identifier element 29 which is configured as an RFID transponder is within communicating range, it will be activated and supplied inductively with power by the signal of the sender/receiver unit 30. In response to the signal of the second identifier element 30, the first element 29 which is now activated and inductively supplied with power returns a data stream, which contains among other things an identification number, to the second identifier element 30 which passes the data on to the controller unit for an automatic recognition and initialization of the dosage-metering unit 8 containing a first identifier element 29. The controller unit contains data regarding the place of the dosage-metering unit 8, i.e. in which of the dosage-metering units it is installed, as well as system- and user-specific data that were stored at an earlier time in the read/write memory of the first identifier element 29.

A titration system according to this embodiment is designed so that a dosage-metering unit 8 with a first identifier element 29 can also be moved through more than one docking device 8 (see also FIGS. 1 and 2) before the dosage-metering device 8 is locked, preferably by hand, into a specific docking device 7, with each docking device being associated with a second identifier element 30. It is therefore preferred to operate the at least one identifier element 30 in the at least one docking device of a titration system in such a way that it sends an interrogation signal only at certain time intervals, so that a dosage-metering unit 8 which is moved through a docking device or happens to be nearby is not instantly recognized and initialized. If a positive reaction is received from a specific first identifier element 29 in response to more than one interrogation signal of a specific second identifier element 30, this indicates that the dosage-metering device 8 carrying the specific first identifier element 29 is seated in the docking device 7 with the specific second identifier element 30, rather than just being pushed through that docking device. Preferably, a dosage-metering unit 8 carrying a first identifier element 29 is initialized only after at least two positive responses to the interrogation signal of a second identifier element 30 have occurred. The time interval for sending the interrogation signal is generally between one second and a few minutes.

The initialization of a dosage-metering unit 8 carrying a first identifier element 29 includes the transmission of data and information from the first identifier element 29 to the second identifier element 30 and further to the controller unit, where the data and information are registered and processed by the software program of the titration system which is implemented in the controller unit. After a dosage-metering unit has been positively recognized, the user receives a message regarding the fact that a new dosage-metering unit has been joined to the titration system and regarding the properties of the dosage-metering unit, which have been determined from the information and data transmitted to the controller unit. These properties include in particular the system- and user-specific information described above, which is stored in the memory of the first identifier element.

The user has the option to confirm this message or, for example if the dosage-metering unit is not yet connected to the desired docking device, to reject the message. After the dosage-metering unit has been initialized, the user has the option to modify the user-specific data and information.

A dosage-metering unit is designed in such a way that it can be connected and locked to any docking device of a titration system. The fact that a connection has been made between a docking device and a dosage-metering device is confirmed through the interaction between the first identifier element 29 of the dosage-metering unit and the second identifier element 30 of the docking device.

FIGS. 5a to 5e illustrate different designs for the docking devices shown and described through FIGS. 1 to 4. Each of the FIGS. 5a to 5e represents a top view of a titration module 13 or a base module 1 as described in the context of FIGS. 1 to 4, with the titration module 13 as well as the base module 1 being shown in a strongly simplified schematic representation and not to scale. The dotted lines indicate the possibility for connecting further titration modules and/or a base module.

FIG. 5a shows a top view of a titration module 13 with a docking device 34 which extends like a channel over the entire length of the titration module 34. A dosage-metering unit can be slid into the docking device 34 from either side. If several titration modules that are designed in this manner are joined together, their docking devices form a continuous channel, so that a dosage-metering unit can be slid through one or more titration modules into any of the docking devices 34 (see also FIG. 1).

FIG. 5b shows a titration module 13 with a docking device 35 that is open only towards one side, so that a dosage unit can only be slid into, but not through the docking device 35. The channel-like recess of the docking device 35 runs perpendicular to the row of titration modules which are arranged side-by-side and is configured as a recess which is delimited on three sides by the housing of the titration module 13. This design of the docking device 35 is particularly advantageous in titration systems with several titration modules, as the dosage-metering units can be exchanged independently of each other.

FIG. 5c shows a titration module 13 with a docking device 36 designed in a T-shaped configuration, so that it combines the advantages of the docking devices 34, 35 that are shown in FIGS. 3a and 3b, respectively. Particularly in titration systems with several titration modules, a titration module 13 of this design can be installed for example in the middle, so that an exchange of dosage-metering units is possible in more than one place, specifically in a titration module that is not at the end of the row, and the side-by-side arrangement of titration modules does not impede the insertion or removal of a dosage-metering unit.

FIG. 5d represents a top view of a titration module 13 with a docking device 37 configured so that a dosage-metering unit can be set in place from above. Like the docking device 35 of FIG. 5b, the docking device 37 cannot work together with the docking devices of other titration modules.

FIG. 5e shows a base module 1 with a two-part docking device. The part 38 of the docking device, which faces towards a further titration module which can be connected, is designed somewhat narrower than the part 39 that adjoins the part 38 on the side that faces away from the titration module. The design of the part 38 is analogous to the design of the docking device shown in FIG. 5a. The part 39 can be used as parking position for a dosage-metering unit that is not in operation at the moment, and also as exchange position for dosage-metering units, as the part 39 is designed so that a dosage-metering unit can be for example set into or taken out of the docking device from above. This two-part configuration is particularly advantageous if a titration module with a single docking device is used consecutively with two dosage-metering units for example for back titrations, because the temporarily unused dosage-metering unit can be put in the parking position.

A titration system can have a base module and at least one titration module, wherein the base module and/or a titration module can either have docking devices of the same kind or of different kinds.

A titration system according to this embodiment can be adapted to the individual needs of the user in an arrangement where each of the components and dosage-metering units that can be connected to the titration system has a means of being recognized, for example an RFID tag which works together with an RFID receiver, or a microcontroller containing a unique identification and a non-volatile read/write memory through which each component and/or each dosage-metering unit can be unequivocally identified and its position within the titration system after the completed connection can be registered.

Such recognition means can be connected to a multitude of components, for example to different sensors, to a stirrer, one or more sample changers, supply containers, an external processing unit such as an appropriately equipped computer, or also to other analyzer instruments and/or peripheral instruments, which may include a balance for determining the sample weight as well as a printer.

A means of recognition includes a microcontroller, a non-volatile memory and a unique identification in the form of a serial number or a special I.D. chip. The means of recognition is designed so that it can be connected to the controller unit through a plug connection that is suitable to transmit power as well as data.

It is preferred to use RFID transponders as identifier elements for the dosage-metering units in conjunction with corresponding sender/receiver units. A titration system according to this embodiment can have for example a barcode and barcode reader as identifier elements and/or a maxi code (a two-dimensional machine-readable code) with a corresponding reader device. The identifier elements can be arranged at any desired location in the housing of the dosage-metering unit or in the docking device, as long as they are able to communicate with each other.

The dosage-metering device and at least one further component can be connected to the titration system, as well as recognized by the latter and initialized, during operation. It is also possible to remove the dosage-metering device again from the titration system, an operation that is likewise automatically recognized. The automatic recognition occurs through suitable recognition means.

Sample changers of the known state of the art include for example revolving turntables with several holders for sample containers which are moved alongside a stationary titration arm.

A CAN bus system allows a nearly unlimited number of components to be connected to a base module. The number of titration modules in a titration system is preferably between one and ten.

The base module and/or at least one titration module can include at least one docking device, but titration systems are also possible which have a base module and/or at least one titration module without a docking device or with more than one docking device.

The controller device can be incorporated in the base module as well as in an external entry- and/or output unit. Furthermore, a controller unit that is integrated in the base module can have an Ethernet connection and thus be controlled by an entry- and/or output unit that is spatially separated from the titration system.

What is claimed is:

1. A titration system comprising:
a base module;
a controller unit containing a processor with an operating program,
a first component, comprising an interchangeable first dosage-metering unit;
at least one further component, comprising at least one of: a further dosage-metering unit, a sensor, a plug-in card, and a titration module, each component releasably connected electronically to the controller unit;
a means for electronically identifying each component to the controller unit with a unique identifier,
a means for establishing and updating a configuration of the components automatically while the titration system is ready for or in operation, the configuration being stored in the operating program.

2. The titration system of claim 1, wherein:
the at least one further component comprises at least one of: a stirrer, a sample changer, a supply container, an external processing unit, an analyzer instrument and a peripheral instrument such as a balance or a printer.

3. The titration system of claim 2, wherein:
the electronic identifying means comprises a read/write memory into which system-specific and/or user-specific data is entered.

4. The titration system of claim 2, wherein:
the electronic identifying means comprises a first identifier element connected to one of the components and a second identifier element connected to the controller unit, the first identifier element adapted to wirelessly transmit the unique identifier to the second identifier element.

5. The titration system of claim 4, wherein:
the first identifier element is a passive RFID transponder and the second identifier element is a corresponding sender and/or receiver unit.

6. The titration system of claim 1, wherein:
each component is electronically connected to the controller unit in a series or parallel connection that is either direct or indirect through another component.

7. The titration system of claim 1, wherein:
the controller unit comprises an interface for transmitting at least one of: data and power.

8. The titration system of claim 7, wherein:
the interface is selected from the group comprising: an RS232 interface, a USB interface, an Ethernet connection, a TTL interface, a CAN bus connection, a current- and/or voltage connection, a parallel port, a serial port, and a connection for a sensor, a measuring probe and/or a peripheral instrument.

9. The titration system of claim 7, wherein:
each component is electronically connected to the controller unit through a suitable interface.

10. The titration system of claim 1, further comprising:
a docking device and a drive mechanism for the dosage-metering unit, the docking device and drive mechanism communicated to at least one of: the base module and the titration module, if present.

11. The titration system of claim 10, wherein:
the further components comprise at least two titration modules, arranged side-by-side and mechanically connectable to each other such that the docking devices of the respective titration modules form a common channel that seats and/or slidingly receives the dosage-metering units.

12. The titration system of claim 1, further comprising:
a display, electronically connected to the controller unit.

13. The titration system of claim 12, wherein:
the controller unit is arranged in the base module.

14. The titration system of claim 1, wherein:
the controller unit is arranged in the base module.

15. A method of operating a titration system comprising a base module, a controller unit containing a processor with an operating program, a first component that is an interchangeable first dosage-metering unit and at least one further component, each component having a releasable electronic connection to the controller unit, the method comprising the steps of:
providing each component with a means of electronically identifying the component to the controller unit with a unique identifier;
establishing a virtual configuration of the components upon start-up, the virtual configuration corresponding to a physical configuration of the components obtained by interrogating each physically configured component to identify the component as present through reception of the unique identifier or absent through lack of reception of the unique identifier, and storing the virtual configuration in an operating program in the controller unit;
changing the physical configuration while the titration system is ready for or in operation by at least one of: removing at least one of the physically configured components and adding at least one component not presently physically configured;
interrogating each physically configured component to identify the component as present through reception of the unique identifier or absent through lack of reception of the unique identifier; and
updating the virtual configuration to correspond to the physical configuration determined in the interrogation step.

16. The method of claim 15, further comprising the step of:
displaying, on a display means of the titration system, a notice requesting a user decision about any impending change in the physical configuration,
wherein the displaying step is performed prior to the interrogating step.

17. The method of claim 16, wherein:
the reception of the unique identifiers by the controller unit from the physically configured components during the interrogating step is achieved through either a wire-bound or a wireless communications means.

18. The method of claim 17, wherein:
the interrogating and updating steps are controlled and/or regulated by the operating program.

19. The method of claim 15, wherein:
the reception of the unique identifiers by the controller unit from the physically configured components during the interrogating step is achieved through either a wire-bound or a wireless communications means.

20. The method of claim 15, wherein:
the interrogating and updating steps are controlled and/or regulated by the operating program.
